(19) United States
(12) Patent Application Publication
(10) Pub. No.: US 2015/0327882 A1
(43) Pub. Date: Nov. 19, 2015

(54) ULTRASONIC DEVICE FOR CUTTING AND COAGULATING

(71) Applicant: Ethicon Endo-Surgery, Inc., Cincinnati, OH (US)

(72) Inventors: Stephen J. Balek, Springboro, OH (US); William D. Dannaher, Cincinnati, OH (US); Carl J. Draginoff, JR., Mason, OH (US); Cory G. Kimball, Hamilton, OH (US); David L. Kohn, Edgewood, KY (US); Galen C. Robertson, Apex, NC (US); Kip M. Tupp, New Richmond, OH (US); Eric B. Smith, Cincinnati, OH (US); Richard C. Smith, Milford, OH (US); Karalyn R. Tellio, Cincinnati, OH (US); Nabeel M. Jadeed, Cincinnati, OH (US)

(21) Appl. No.: 14/806,762

(22) Filed: Jul. 23, 2015

Related U.S. Application Data
(63) Continuation of application No. 13/457,887, filed on Apr. 27, 2012.
(60) Provisional application No. 61/479,901, filed on Apr. 28, 2011.

(51) Int. Cl. A61B 17/32; A61N 7/00

(52) U.S. Cl. CPC A61B 17/320068; A61N 7/00; A61B 2017/00424

(57) ABSTRACT

An ultrasonic assembly that is configured to permit selective cutting, coagulation, and fine dissection required in fine and delicate surgical procedures. The balanced blade provides a rounded distal end and concave edges to promote fine dissection and cutting in a variety of surgical procedures. The blade is curved for improved visibility at the blade tip and is designed to provide a multitude of tissue effects: coagulation, cutting, dissection, spot coagulation, tip penetration and tip scoring. The assembly features hand activation configured to provide an ergonomical grip and operation for the surgeon. The assembly further features user selectable blade rotation. A finger switch is placed in the range of the natural axial motion of the user's index finger, whether gripping the surgical instrument right-handed or left handed.

### REFERENCE TO RELATED APPLICATIONS

This application claims priority to U.S. Provisional Patent Application Ser. No. 61/479,901 filed Apr. 28, 2011 entitled “Ultrasonic Device for Cutting and Coagulating.”

### FIELD OF THE ULTRASONIC DEVICE

The present ultrasonic device generally relates to ultrasonic surgical systems and, more particularly, to an ultrasonic device that allows surgeons to perform cutting and coagulation in orthopedic procedures.

### BACKGROUND OF THE ULTRASONIC DEVICE

Ultrasonic surgical instruments are finding increasingly widespread applications in surgical procedures by virtue of the unique performance characteristics of such instruments. Depending upon specific instrument configurations and operational parameters, ultrasonic surgical instruments can provide substantially simultaneous cutting of tissue and homeostasis by coagulation, desirably minimizing patient trauma. The cutting action is typically realized by an end-effector, or blade tip, at the distal end of the instrument, which transmits ultrasonic energy to tissue brought into contact with the end-effector. Ultrasonic instruments of this nature can be configured for open surgical use, laparoscopic or endoscopic surgical procedures including robotic-assisted procedures.

However, the advanced energy instruments currently available are not designed specifically for orthopedic surgery procedures. They lack the comfort and versatility required for such procedures.

Some surgical instruments utilize ultrasonic energy for both precise cutting and controlled coagulation. Ultrasonic energy cuts and coagulates by using lower temperatures than those used by electrosurgery. Vibrating at high frequencies (e.g. 55,500 times per second), the ultrasonic blade denatures protein in the tissue to form a sticky coagulum. Pressure exerted on tissue with the blade surface collapses blood vessels and allows the coagulum to form a hemostatic seal. The precision of cutting and coagulation is controlled by the surgeon's technique and adjusting the power level, blade edge, tissue traction and blade pressure.

It would be desirable to provide an ultrasonic surgical instrument that overcomes some of the deficiencies of current instruments available for use in orthopedic and other surgical procedures. The ultrasonic surgical instrument described herein overcomes those deficiencies.

### BRIEF DESCRIPTION OF THE FIGURES

The novel features of the ultrasonic device are set forth with particularity in the appended claims. The ultrasonic device itself, however, both as to organization and methods of operation, may best be understood by reference to the following description, taken in conjunction with the accompanying drawings in which:

FIG. 1 is a perspective view of the present ultrasonic device;

FIG. 2 is an assembly view of one expression of the present ultrasonic device;

FIG. 3 is a plan view of a first expression of a waveguide and blade design in accordance with the present ultrasonic device;

FIG. 4 is an elevation view of the first expression of a waveguide and blade design in accordance with the present ultrasonic device;

FIG. 5 is an exploded plan view of the blade design of the first expression in accordance with the present ultrasonic device;

FIG. 6 is an exploded elevation view of the blade design of the first expression in accordance with the present ultrasonic device;

FIG. 7 is a cut-away view of the cross section of the blade design of the first expression in accordance with the present ultrasonic device;

FIG. 8 is a plan view of a second expression of a waveguide and blade design in accordance with the present ultrasonic device;

FIG. 9 is an elevation view of the second expression of a waveguide and blade design in accordance with the present ultrasonic device;

FIG. 10 is an exploded plan view of the blade design of second expression in accordance with the present ultrasonic device;

FIG. 11 is an exploded elevation view of the blade design of the second expression in accordance with the present ultrasonic device;

FIG. 12 is a cut-away view of the cross section of the blade design of the second expression;

FIG. 13 is a frontal view of the blade design of the second expression of the present ultrasonic device;

FIG. 14 is a perspective view of an embodiment of the present ultrasonic device with coatings to denote different areas of the blade;

FIG. 15A is a perspective view of a sheath and transducer;

FIG. 15B is a cutaway view of the present ultrasonic device rotation and locking mechanism;

FIG. 16A is a perspective view of a waveguide cover;

FIG. 16B is an elevation view of an alternate expression of a waveguide cover;

FIG. 17A is a perspective view of another expression of a waveguide cover;

FIG. 17B is an elevation view of another expression of a waveguide cover; and

FIG. 17C is a perspective view of another expression of a waveguide cover.

### DETAILED DESCRIPTION OF THE ULTRASONIC DEVICE

Before explaining the present ultrasonic device in detail, it should be noted that the ultrasonic device is not limited in its application or use to the details of construction and arrangement of parts illustrated in the accompanying drawings and description. The illustrative embodiments of the ultrasonic device may be implemented or incorporated in other embodiments, variations and modifications, and may be practiced or carried out in various ways. Further, unless otherwise indicated, the terms and expressions employed herein have been chosen for the purpose of describing the illustrative embodiments of the present ultrasonic device for the convenience of the reader and are not for the purpose of limiting the ultrasonic device.

Further, it is understood that any one or more of the following-described embodiments, expressions of embodiments, examples, etc. can be combined with any one or more of the other following-described embodiments, expressions of embodiments, examples, etc.

The present ultrasonic device is particularly directed to an improved ultrasonic surgical instrument, which is configured for effecting tissue dissecting, cutting and/or coagulation during surgical procedures, such as orthopedic or neurologic surgery. The instrument is configured to facilitate soft tissue access in open, multi-level posterior spine procedures. Disclosed is a hemostatic blade to dissect muscle and tough tissues such as facia and tendon and dissect tissues off of bone such as periosteum and tendon attachments. The present apparatus is configured for use in open surgical procedures, but has applications in other types of surgery, such as laparoscopic and other minimally invasive surgical procedures. Versatile use is facilitated by selective use of ultrasonic energy. When ultrasonic components of the apparatus are inactive, tissue can be manipulated, as desired, without tissue cutting or damage. When the ultrasonic components are activated the ultrasonic energy provides for both tissue cutting and coagulation.

Further, the present ultrasonic device is disclosed in terms of a blade-only instrument. This feature is not intended to be limiting, as the embodiments disclosed herein have equal application in clamp coagulator instruments as are exemplarily disclosed in U.S. Pat. Nos. 5,873,873 and 6,773,444.

As will become apparent from the following description, the present surgical apparatus is particularly configured for disposable use by virtue of its straightforward construction. As such, it is contemplated that the apparatus be used in association with an ultrasonic generator unit of a surgical system, whereby ultrasonic energy from the generator unit provides the desired ultrasonic actuation for the present surgical instrument. It will be appreciated that surgical instrument embodying the principles of the present ultrasonic device can be configured for non-disposable or multiple use, and non-detachably integrated with an associated ultrasonic generator unit.

Some current designs of ultrasonic devices utilize a foot pedal to energize the surgical instrument. The surgeon operates the foot pedal to activate a generator that provides energy that is transmitted to the cutting blade while simultaneously applying pressure to tissue with an ultrasonic blade for cutting and coagulating tissue. Key drawbacks with this type of instrument activation include the loss of focus on the surgical field while the surgeon searches for the foot pedal, the foot pedal getting in the way of the surgeon's movement during a procedure and surgeon leg fatigue during long cases.

Various means have been disclosed for curved end effector balancing, which include repositioning the mass along the end effector. The drawbacks of such methods are i) high stresses in the curved region, which makes the end effector more prone to fracture if it comes in contact with metal during surgery; ii) a shorter active length, which limits the vessel size that can be operated on, (the active length is defined as the length from the distal end of the blade to where the displacement is one half of the displacement at its distal end); and/or iii) the inability to separately balance orthogonal displacements.

The present ultrasonic surgical instrument overcomes the disadvantages of prior instruments used in orthopedic or neurologic surgery by providing a versatile transmission assembly for cutting and coagulation. The present ultrasonic instrument further provides the surgeon the ability to selectively rotate the transmission assembly facilitating ergonomic use of the ultrasonic instrument.

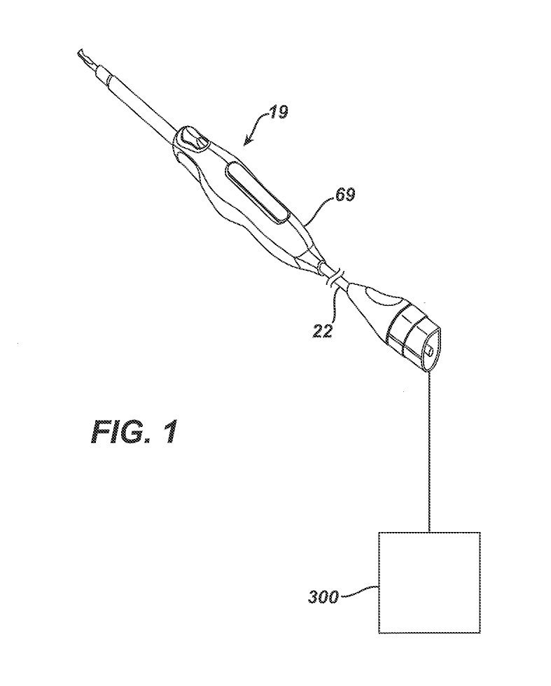

With specific reference now to FIG. 1, an embodiment of a surgical system, including an ultrasonic surgical instrument 19 in accordance with the present ultrasonic device, is illustrated. The surgical system 19 includes an ultrasonic generator 300 connected to an ultrasonic transducer 50 via cable 22 (not shown to scale), and an ultrasonic surgical instrument 19. It will be noted that, in many applications, the ultrasonic transducer 50 is also traditionally referred to as a “hand piece assembly” or “handpiece” because in some surgical instruments a surgeon may grasp and manipulate the ultrasonic transducer 50 during various procedures and operations. A suitable generator 300 is the GEN04 or GEN11 sold by Ethicon Endo-Surgery, Inc. of Cincinnati, Ohio. A suitable transducer is disclosed in co-pending U.S. patent application filed on Oct. 10, 2006, Ser. No. 11/545,784, entitled MEDICAL ULTRASOUND SYSTEM AND HANDPIECE AND METHODS FOR MAKING AND TUNING, the entire contents of which are herein incorporated by reference. Although a remote generator and power supply is disclosed, it is contemplated that the device 19 may incorporate a generator and power supply for tetherless operation, as is disclosed in U.S. patent application Ser. No. 13/275,495, which is herein incorporated by reference.

Ultrasonic transducer 50 and an ultrasonic waveguide 80 together provide an acoustic assembly of the present surgical system 19, with the acoustic assembly providing ultrasonic energy for surgical procedures when powered by generator 300 or in the tetherless embodiment, an on-board power supply and generator. The acoustic assembly of surgical instrument 19 generally includes a first acoustic portion and a second acoustic portion. In the present embodiment, the first acoustic portion comprises the ultrasonically active portions of ultrasonic transducer 50, and the second acoustic portion comprises the ultrasonically active waveguide 80 and blade 79. Further, in the present embodiment, the distal end of the first acoustic portion transducer 50 is operatively coupled to the proximal end of the waveguide 80 by, for example, a threaded connection.

The ultrasonic surgical instrument 19 includes a multi-piece handle assembly 69 (comprised of handle shroud halves 69A and 69B) adapted to isolate the operator from the vibrations of the acoustic assembly contained within transducer 50. The handle assembly 69 can be shaped to be held by a user in a conventional manner, but it is contemplated that the present ultrasonic surgical instrument 19 principally be grasped and manipulated in a pencil-like arrangement provided by a handle assembly 69 of the instrument, where the handle 69 is adapted to rest on the top of the hand surface between the index finger and thumb and to be grasped by the thumb and middle finger. The instrument is further provided with a switch or trigger on top of the instrument 19 adapted to be activated by the index finger when held in this fashion.

While a multi-piece handle assembly 69A, 69B is illustrated, the handle assembly 69 may comprise a single or unitary component. The proximal end of the ultrasonic surgical instrument 19 receives and is fitted to the distal end of the ultrasonic transducer 50 by insertion of the transducer into the handle assembly 69. The ultrasonic surgical instrument 19 may be attached to and removed from the ultrasonic transducer 50 as a unit. Transducer 50 and handle 69 may be adapted to permit transducer 50 to rotate within handle 69 and it is contemplated that transducer 50 may be non-detachably provided in handle 69. The elongated transmission assembly 80 of the ultrasonic surgical instrument 19 extends orthogonally from the instrument handle assembly 69.

The handle assembly 69 may be constructed from a durable plastic, such as polycarbonate or a liquid crystal polymer. It is also contemplated that the handle assembly 69 may alternatively be made from a variety of materials including other plastics, ceramics or metals. Traditional unfilled thermoplastics, however, have a thermal conductivity of only about 0.20 W/m° K (Watt/meter-° Kelvin). In order to improve heat dissipation from the instrument, the handle assembly may be constructed from heat conducting thermoplastics, such as high heat resistant resins liquid crystal polymer (LCP), Polyphenylene Sulfide (PPS), Polyetheretherketone (PEEK) and Polysulfone having thermal conductivity in the range of 20-100 W/m° K. PEEK resin is a thermoplastics filled with aluminum nitride or boron nitride, which are not electrically conductive. The thermally conductive resin helps to manage the heat within smaller instruments.

Activation board assembly 215 comprises a pushbutton assembly 210, a circuit board assembly 220, a first pin 210A and a second pin 210B. Switch assembly 215 is configured in a rocker arrangement and is supported within handle assembly 69 by way of corresponding supporting mounts 230A and 230B in housing portions 69A and 69B.

Switch 210 is provided with pins 210A and 210B that mechanically contact dome switches 220A and 220B. For the selective activation of ultrasonic energy, circuit board 220 electrically connects to the proximal end of transducer 50. Proximal end of transducer 50 is provided with a plug that is in electrical communication with transducer 50 as well as switch 210. Cable 22 may be provided with a plug that mates with transducer 50 plug providing electrical communication with transducer 50 plug which, in turn, connects to generator 300. In another expression, cable 22 may be integrally attached to transducer 50 and switch 210. As set forth above, switch 210 is pivotally attached to housing 69 to permit the surgeon to selectively energize instrument 19 with an index finger when held in a pencil-like arrangement. When assembled, trigger 210 pivotally attaches to housing 69 and contact surfaces 210A and 210B mechanically engage dome switches 220A and 220B, respectively. Ridges (not shown) on the switch 210 provide an interface between the user and switch 210 and are adapted to provide as much surface area for the user to depress in order to activate the instrument. The ridges may be of different shapes and sizes to give the surgeon tactile feel of which switch is associated with a high power application or low power application.

Circuit board 220 provides for the electro-mechanical interface between pushbuttons switch 210 and the generator 300 via transducer 50. Flex circuit comprises two dome switches 220A and 220B that are mechanically actuated by depressing switch 210 in the Z-axis direction. Dome switches 220A and 220B are electrical contact switches, that when depressed provide an electrical signal to generator 300, as is known and understood in the art. Circuit board 220 generally sits within a channel of housing providing support for the dome switches during operation.

As is readily apparent, by depressing switch 210 the corresponding contact surfaces 210A or 2108 depress against corresponding dome switches 220A or 220B to activate a circuit. When the surgeon depresses switch 210 (switch 210 pivots about a central point permitting the proximal or distal portion to travel in the Z-axis), the generator will respond with a certain energy level, such as a maximum (“max”) power setting; when the surgeon rocks switch 210 in the opposite direction, the generator will respond with a certain energy level, such as a minimum (“min”) power setting, which conforms to accepted industry practice for pushbutton location and the corresponding power setting.

Switch 210 location and manner of actuation when held in a pencil-like fashion reduces stress on the surgeon's fingers and hand and allows the fingers to actuate instrument 19 in a more ergonomic position preventing stresses at the hands and wrists. The switch 210 location also allows comfortable switch 210 activation in less than optimal hand positions, which surgeons often encounter throughout a typical procedure.

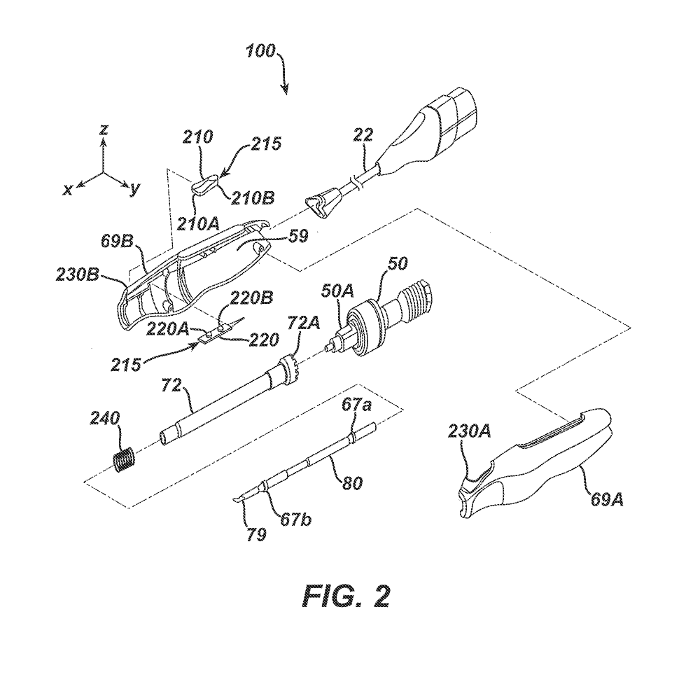

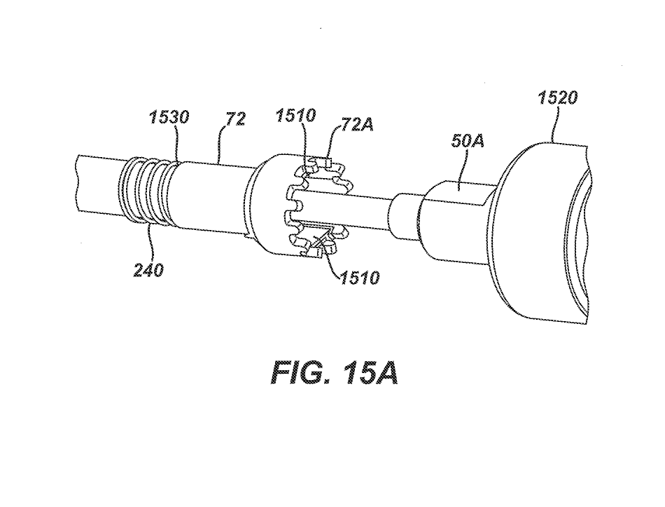

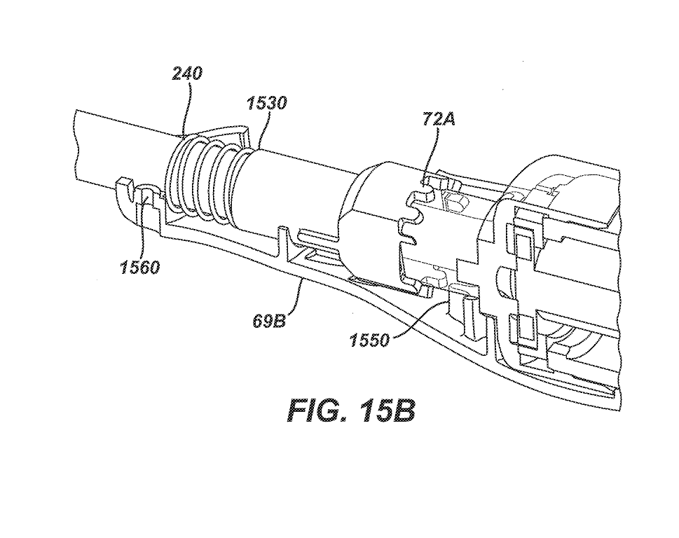

Still referring to FIG. 2, instrument 19 may be further provided with a waveguide sheath 72 to isolate the surgeon from waveguide 80. Sheath 72 is adapted to shield waveguide 80 during activation. Sheath 72 is configured with teeth 72A (shown in FIG. 15A) that mate with handle 69 locking or stop teeth (described more fully herein). Transducer 50 may be configured with distal flats 50A that mate with flats disposed within sheath 72 proximal end to permit rotation of waveguide 72, sheath 72 and transducer 50 as a single unit. Spring 240 is provided between handle 69 and waveguide sheath 72 to bias sheath 72 into fixed positions relative to handle 69, preventing inadvertent rotation of sheath 72, waveguide 80 and transducer 50 and is more fully described herein.

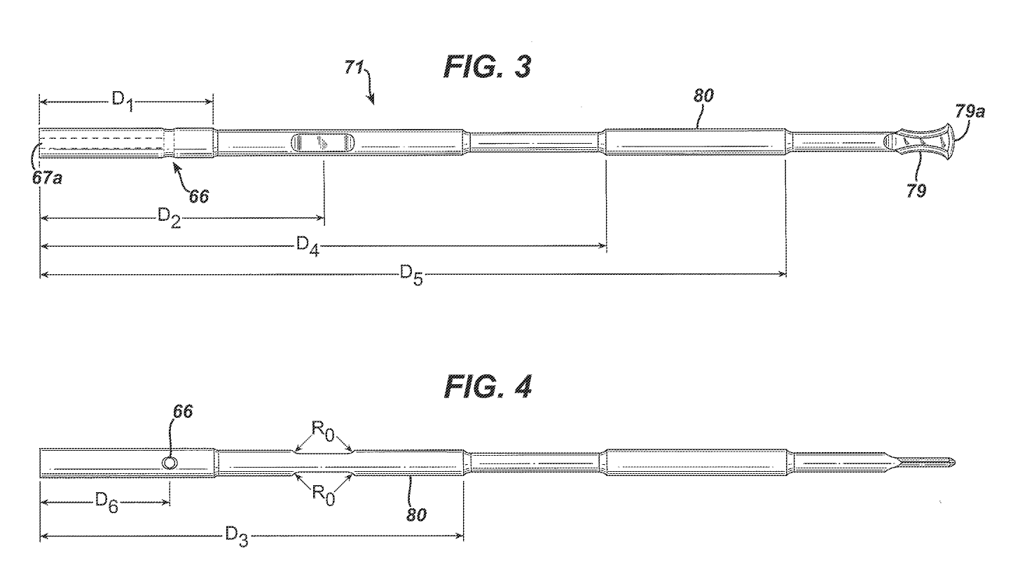

With reference to FIGS. 3-13, the transmission assembly 71 includes a waveguide 80 and a blade 79. It will be noted that, in some applications, the transmission assembly is sometimes referred to as a “blade assembly”. The waveguide 80, which is adapted to transmit ultrasonic energy from transducer 50 to the tip of blade 79 may be flexible, semi-flexible or rigid. The waveguide 80 may also be configured to amplify the mechanical vibrations transmitted through the waveguide 80 to the blade 79 as is well known in the art. The waveguide 80 may further have features to control the gain of the longitudinal vibration along the waveguide 80 and features to tune the waveguide 80 to the resonant frequency of the system. In particular, waveguide 80 may have any suitable cross-sectional dimension. For example, the waveguide 80 may be tapered at various sections to control the gain of the longitudinal vibration, as discussed more fully herein.

Ultrasonic waveguide 80 may, for example, have a length substantially equal to an integral number of one-half system wavelengths (nλ/2). The ultrasonic waveguide 80 and blade 79 may be preferably fabricated from a solid core shaft constructed out of material, which propagates ultrasonic energy efficiently, such as titanium alloy (i.e., Ti-6Al-4V), aluminum alloys, sapphire, stainless steel or any other acoustically compatible material.

Ultrasonic waveguide 80 may further include at least one radial hole or aperture 66 extending therethrough, substantially perpendicular to the longitudinal axis of the waveguide 80. The aperture 66, which may be positioned at a node, is provided in combination with a vent aperture 66a to ensure proper EtO sterilizing when waveguide 80 is threaded to transducer in a disposable transducer device. Proximal o-ring 67a and distal o-ring 67b (see FIG. 2) are assembled onto transmission assembly 71 near the ultrasonic nodes of waveguide 80, as is known in the art.

Blade 79 may be integral with the waveguide 80 and formed as a single unit. In an alternate expression of the current embodiment, blade 79 may be connected by a threaded connection, a welded joint, or other coupling mechanisms. The distal end of blade 79, or blade tip 79a, is disposed near an anti-node in order to tune the acoustic assembly to a preferred resonant frequency fₒ when the acoustic assembly is not loaded by tissue. When ultrasonic transducer 50 is energized the blade tip 79a is configured to move substantially longitudinally (along the x axis) in the range of, for example, approximately 10 to 500 microns peak-to-peak, and preferably in the range of about 20 to about 200 microns at a predetermined vibrational frequency fₒ of, for example, 55,500 Hz. Blade tip 79a also preferably vibrates in the Z-axis at about 1 to about 10 percent of the motion in the X-axis.

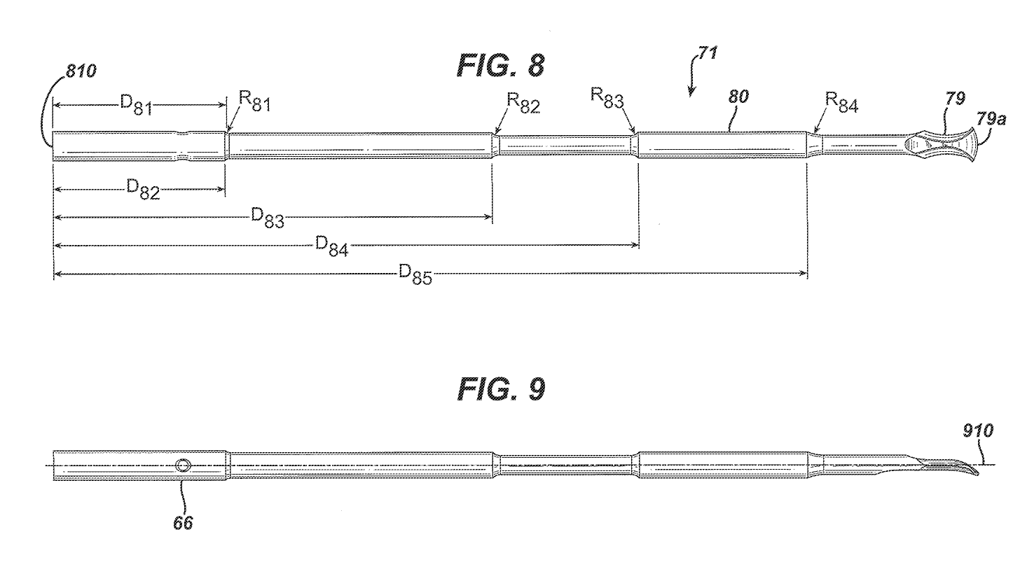

FIGS. 3-7 illustrate a straight blade 79, and FIGS. 8-13 illustrate a curved blade 79 that matches vertebral curvature to maximize the ability of the harmonic blade to remove muscle, connective tissue, and fascia from bone. Blade 79 is configured in a “battle axe” or double hook shape to provide multiple cutting and dissection surfaces. Blade 79 edges are beveled to promote dissection of tissues encountered in orthopedic procedures and to further provide faster cutting when ultrasonic energy is applied to blade 79. In some types of orthopedic surgery, e.g. spine surgery, the operative incision may be small permitting access to only one or two instruments. The versatility of the ultrasonic device 19 provides ergonomic dissection, cutting and coagulation in a single instrument.

Referring now to FIGS. 3-7, a first expression of transmission assembly 71 is shown. As stated above, waveguide 80 is provided with a series of features to amplify the longitudinal excursion of blade 79. As shown in FIG. 3, waveguide 80 has a preferred overall length of about 5.314 inches. A first gain step, measured from proximal end 67a, is preferably located about 1.010 inches from 67a and is denominated D₁ having a preferred diameter of about 0.170 inches. A second gain step, shown as a notch in waveguide 80, is centered at about 1.25 inches from 67a, denominated distance D₂ and is approximately 0.366 inches in length along the longitudinal axis of waveguide 80 and is formed by cutouts in waveguide 80.

As shown, the second gain step is not a full radius cutout, rather a notch on the top and bottom of wavedguide 80 having radii, R₀ of about 0.063 inches. A third gain step is placed approximately 2.56 inches from 67a and is denominated D₃ in FIG. 4. The diameter of waveguide 80 between D₁ and D₃ is preferably about 0.145 inches. Waveguide 80 increases in diameter at an anti-node preferably located approximately 3.29 inches from 67a and is denominated D₄ and the diameter of waveguide 80 in the D₃ to D₄ section is preferably about 0.110 inches. A final gain step, D₅ is preferably located approximately 4.33 inches from 67a having a diameter between D₄ and D₅ of 0.150 inches. The diameter of waveguide 80 proximal to blade 79 is preferably about 0.110 inches. The transition area between the smaller diameter sections of waveguide 80 and the various gain steps has cutout radii of approximately 0.060 inches.

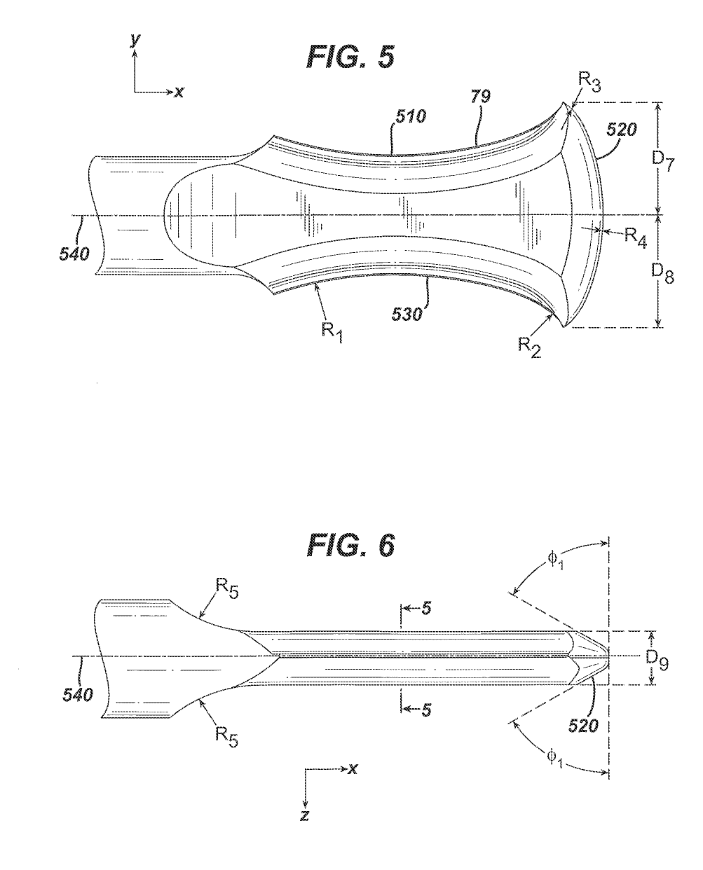

Referring now to FIGS. 5 and 6, the dimensions of blade 79 are shown. As set forth above, blade 79 is adapted for use in orthopedic procedures. The battle axe shape of blade 79 permits a surgeon to use three surfaces, 510, 520 and 530 for dissection, cutting and coagulating and is suited for use in and around vertebrae. Blade 79 may be symmetrical about axis 540 where surface 510 and 530 have nearly identical dimensions and are concave in shape. Surface 520 is rounded in shape and extends distally longer along axis 540 than at its lateral edges.

As shown in FIG. 5, surfaces 510 and 530 are formed from two radii cuts, R₁ and R₂, where R₁ has a preferred radius of about 0.35 inches and R₂ has a preferred radius of about 0.080 inches. Blade 79 distal end 520 is rounded about axis 540 and is defined by radii R₃ and R₄. Radius R₃ has a preferred radius of approximately 0.060 inches and R₄ has a preferred radius of approximately 0.20 inches. The lateral most points of blade 79, as shown in FIG. 5 are preferably about 0.105 inches from axis 540, shown as D₇ and D₈.

FIG. 6 depicts a side view of blade 79. Axis 640 is coextensive with X-axis 540 shown in FIG. 5, and is defined by an X-Y plane as shown in FIG. 5. In one expression of waveguide 80, blade 79 has a thickness D₉ of about 0.050 inches. Blade 79 is further provided with beveled surface 520 to facilitate dissection of tissue from bone and cutting. In one expression, surface 520 is beveled at an angle φ₁, which is, in one expression, is preferably 45°-70° and most preferably 60°. Cross section 5-5 of blade 79 is approximately 0.046 to 0.054 inches. Blade 79 is further provided with waveguide 80 transition cut-outs, R₅, having radii of approximately 0.130 inches.

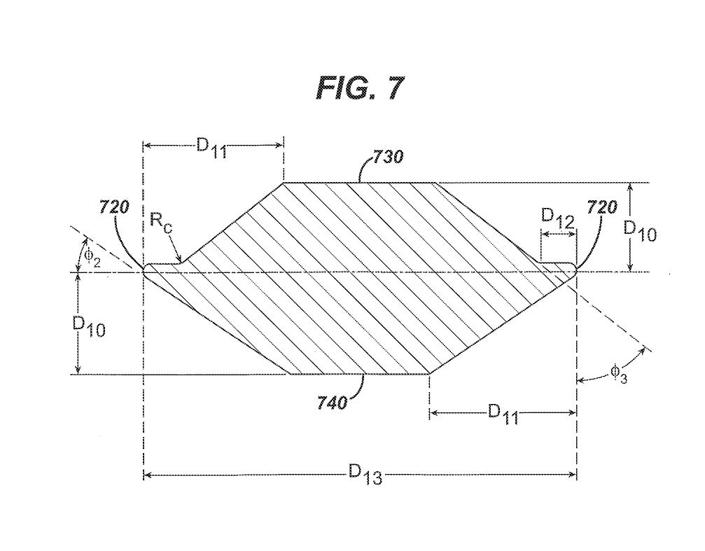

Referring now to FIG. 7, the FIGS. 5 and 6 blade 79 is shown as a cut-away cross section taken at section 5-5 in FIG. 6. Blade 79 has a central top ridge 730 and a central bottom ridge 740. Edges 720 are partially formed by edges beveled away at obtuse angles from central top ridge 730 and central bottom ridge 740.

As shown in FIG. 7, blade 79 has an overall thickness of approximately 0.050 inches comprising 2×D₁₀ of approximately 0.025 inches. The overall width of blade 79 as shown in FIG. 7 and denominated D₁₃ is approximately 0.11 inches. Flange or cutting surface 720 has a width of D₁₂, approximately 0.010 inches, and is formed from radius transition R₆ where R₆ has a preferred radius of about 0.002 inches. Lateral surfaces of blade 79 are defined by beveled angles φ₂ and φ₃ where φ₂ is preferably 30°-40° and most preferably 34° and φ₃ is preferably 35°-45° and most preferably 38.1°. Beveled sections are defined by width D₁₁ of approximately 0.037 inches. Blade 79 top 730 is substantially flat and defines a central top ridge. Blade 79 bottom 740 is substantially flat and defines a central bottom ridge.

Referring now to FIGS. 8 and 9, a second expression of waveguide 80 and 79 is shown. As discussed previously, waveguide 80 is provided with a series of gain steps, as is known in the art. A first gain step is located at distance D₈₂ from waveguide 80 proximal end 810. Distance D₈₂ is preferably 0.997 to 1.003 inches from end 810. Length D₈₂ has a preferred diameter of 0.169 to 0.171 inches. The terminal end of the first gain step transitions distally via radius cutout R₈₁ which has a preferred radius of about 0.032 inches. A second gain step is located at a distance D₈₃ from end 810 and is preferably 2.547 to 2.553 inches from end 810 and has a preferred diameter of 0.149 to 0.151 with a transition radius cut, R₈₂ of approximately 0.063 inches. Waveguide 80 increases in diameter at distance D₈₄ with a radius cut R₈₃ of approximately 0.063 inches, co-located with a wave anti-node as is known in the art. D₈₄ is preferably 3.397 to 3.403 inches from end 810 and has a preferred diameter of 0.109 to 0.111 inches. A third gain step is located at a distance D₈₅ from end 810 having a preferred distance of 4.372 to 4.378 inches formed by radius cut R₈₄ having a radius of about 0.250 inches. Waveguide 80 is provided with through hole 66, as discussed previously.

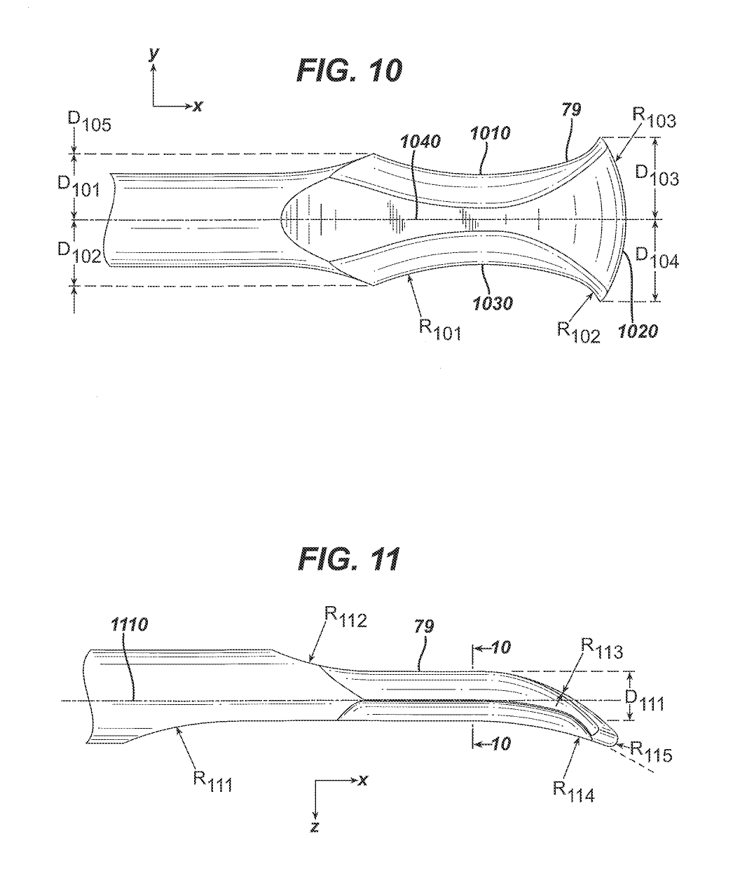

Referring now to FIG. 10, an exploded plan view of the blade 79 of the second expression of waveguide 80 is shown. In this expression, blade 79 is curved away from axis 1110 along the Z-axis, as shown in FIG. 11. Blade 79 curves away from the plane defined by the X-Y axis in FIG. 10 where the Y-axis denoted in FIG. 11 and the Y-axis denoted in FIG. 10 are coextensive in nature and centered along axis 1110.

The curved nature of the blade may provide better visibility and better access to deep spaces in and around the spine or in any other confined operative site. Shaft diameter proximal to blade is denominated by equal distances D₁₀₁ and D₁₀₂, collectively preferably 0.113 to 0.115 inches. As shown in FIG. 10, blade 79 is symmetrical about axis 1040, where lateral surfaces 1010 and 1030 have nearly identical dimensions and are concave in shape. Lateral surfaces 1010 and 1030 are formed by multiple radii cuts denominated R₁₀₁ and R₁₀₂ where R₁₀₁ is preferably approximately 0.350 inches and R₁₀₂ is preferably approximately 0.059 inches. Distal surface 1020 has a rounded end defined by radius R₁₀₃ where R₁₀₃ has a preferred radius of about 0.383 inches. Distal blade width, set forth equal distances D₁₀₃ and D₁₀₄, where D₁₀₃ and D₁₀₄ each measure approximately 0.101 inches. Dimension of proximal end of surfaces 1010 and 1030 is denominated by distance D₁₀₅ and is approximately 0.162 inches.

The curved nature of blade 79 discussed above is depicted in exploded elevation view in FIG. 11. Blade 79 is formed from radii cuts R₁₁₃ and R₁₁₄. Radius R₁₁₄ bends away from central axis 1110 via radius cut R₁₁₄ having a preferred radius of about 0.475 inches. Radius R₁₁₃ has a preferred radius of about 0.250 inches. Blade 79, in this expression, is formed by radii transitions in waveguide 80 denominated R₁₁₂ and R₁₁₃ in FIG. 9. R₁₁₂, in one expression, has a preferred radius of about 0.300 inches and R₁₁₁ has a preferred radius about 0.350 inches. As shown in FIG. 11, blade 79 has a proximal thickness, D₁₁₁ of 0.056 to 0.064 inches.

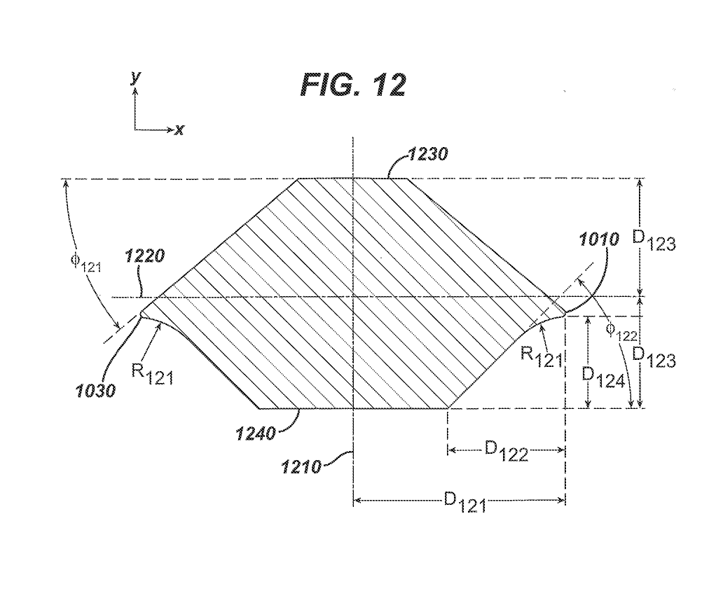

Referring now to FIG. 12, the FIGS. 8 and 9 blade 79 is shown as a cut-away cross section taken at section 10-10 in FIG. 11. Blade 79 has a central top ridge 1230 and a central bottom ridge 1240. Edges 1010 and 1020 are partially formed by beveling away at obtuse angles from central top ridge 1030 and central bottom ridge 1040.

The cross section shown in FIG. 12 is divided equally by axes 1210 in the Y-axis and 1220 in the X-axis. As shown, blade 79 has an overall thickness of approximately 0.060 inches comprising 2×D₁₂₃ of approximately 0.030 inches. The overall width of blade 79 as shown in FIG. 12 and denominated 2×D₁₂₁ is approximately 0.220 inches, where D₁₂₁ is preferably 0.110 inches. Flange or cutting surfaces 1010 and 1020 are formed from radius transition R₁₂₁ where R₁₂₁ has a preferred radius of about 0.002 inches. Lateral surfaces of blade 79 are defined by beveled angles φ₁₂₁ and φ₁₂₂ where φ₁₂₁ is preferably 35°-45°, most preferably 40° and φ₁₂₂ is preferably 40°-50° most preferably 45°. Lower beveled sections are defined by width D₁₂₄ of approximately 0.024 inches.

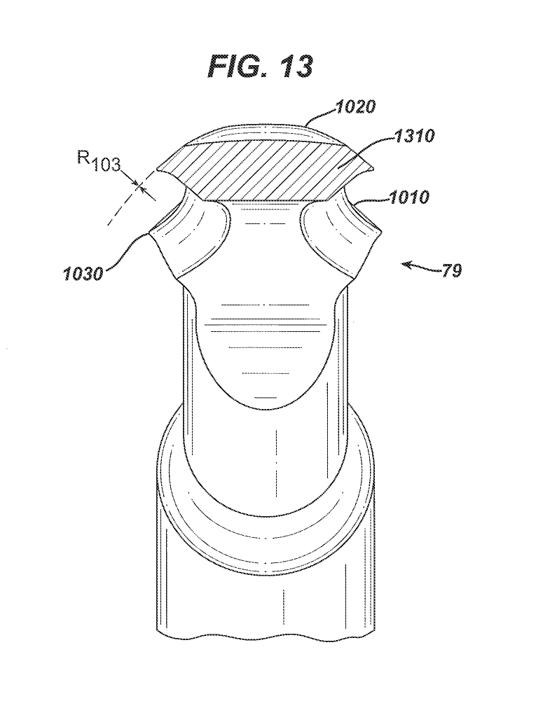

FIG. 13 is a frontal view of the blade 79 design shown in FIGS. 10-13. As discussed previously, distal end of blade 79 is curved as defined by radius R₁₀₃. In one expression, distal end may be further beveled providing an edge 1310 to facilitate dissection and cutting.

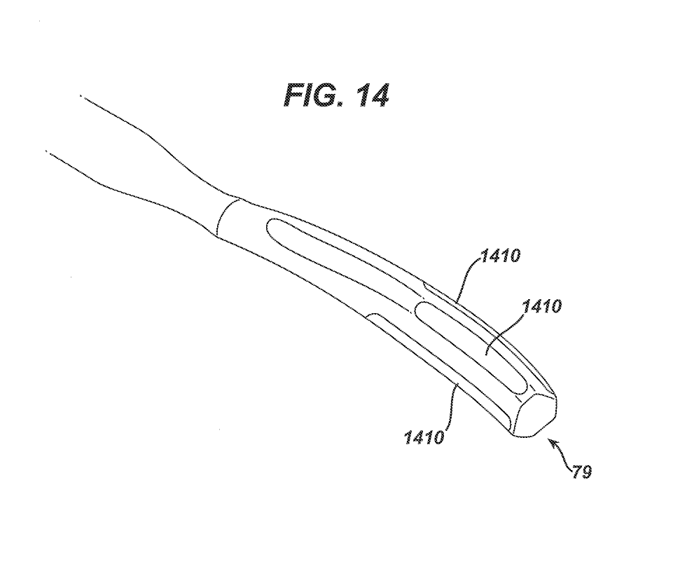

Blade 79 set forth above may be modified with visible markings to facilitate surgeon adoption and ease of use. As shown in FIG. 14, an anodized coating 1410 may be applied to selected surfaces of the blade 79 to make it more apparent to surgeons which area of the blade is most suitable for cutting and coagulating tissue. By anodizing in two different colors it may be easier for surgeons to understand which areas of the blade 79 are best for cutting and how they are different from the areas on the blade 79 that are best for coagulation.

As stated previously, waveguide 80 is positioned within outer sheath 72. The sheath 72 covers the blade from just proximal to the blade 79 to the handle 69. At the distal end there is a seal 67b (see FIG. 2) between the waveguide 80 and sheath 72 to prevent fluid migration up the waveguide 80 and between the waveguide 80 and sheath 72. The area around this seal may heat up to a temperature that would not be comfortable with prolonged skin contact of either the surgeon or the patient. A warning, texture, color, etc. can be used to demarcate where it is safe to touch for long periods and where it isn't (not shown). This may also be used to indicate rotation instructions. There is also a potential to utilize a metal sheath (not shown) on the interior of the plastic sheath to better conduct thermal energy thereby dissipating it over a larger surfer area.

Referring now to FIGS. 15A and 15B, sheath 72 is placed over waveguide 80 and is positioned to expose the proximal end (screw thread) of the waveguide 80. The waveguide 80 threads onto the stud of the transducer 50 that has a nose cone 1520 and torque to spec, this is accomplished by “flats” on the blade and tool to match flat spacing or a tool that uses opposing pins that engage in thru hole 66 that's perpendicular to the axis, as is known in the art.

The sheath 72 proximal end has a circular pattern of gear teeth 72A, and the inner diameter of sheath 72 is sized to fit over the nose cone 1520 of transducer 50. There are opposing flats 1510 on in the inner surface of the sheath 72 that are sized to fit over outer opposing flats 50A on transducer 50 nose cone 1520, and these flats 1510 are sized to “key” the sheath 72 to the nose cone 1520. When the sheath 72 flats 72A are engaged onto the nose cone 1520 flats 50A, and the waveguide 80 has already been torqued to the transducer 50, the entire assembly is then keyed to rotate.

A coil spring 240 is positioned over the sheath 72 and the entire assembly is placed into the right handle shroud 69B. The spring 240 compresses using a rib wall 1560 in the shroud 69B and wall 1530 on the sheath 72. This forces the sheath 72 rearward until the sheath gear teeth 72A engage into the shroud 69B tooth stop 1550. Flats 1510 in sheath 72 and nose cone 1520 have a length greater than the travel of the sheath 72 between shroud wall 1530 and rib wall 1560 allowing flats to remain engaged at all times.

In operation it may be desirable for a surgeon to rotate blade 79 to create different blade 79 positions relative to handle 69. This permits the surgeon to continue to grip ultrasonic device 19 in a pencil-like fashion to promote ergonomic use while simultaneously creating different blade positions that may permit better access to structures in and around an operative site.

To position the blade 79 to the desired angle relative to the handle 69, the user holds the instrument 19 handle assembly 69 in one hand and with the other hand grabs the sheath 72 and pulls outward along a longitudinal axis defined by waveguide 80 and sheath 72, (only the sheath moves along the axis), which compresses the spring 240 and disengages the sheath 72 gear teeth 72A from the shroud 69 stop tooth 1550. The operator is then free to rotate the sheath 72 that also rotates the waveguide 80 and transducer assembly 50 and the blade 79 to the desired blade position. To re-lock the sheath 72, the user simply releases the sheath 72 and the spring 240 biases only the sheath 72 towards stop tooth 1550 until the teeth 72A engage the shroud tooth stop 1550. In other expressions of the present device, multiple stop teeth 1550 may be provided creating more support to prevent inadvertent rotation.

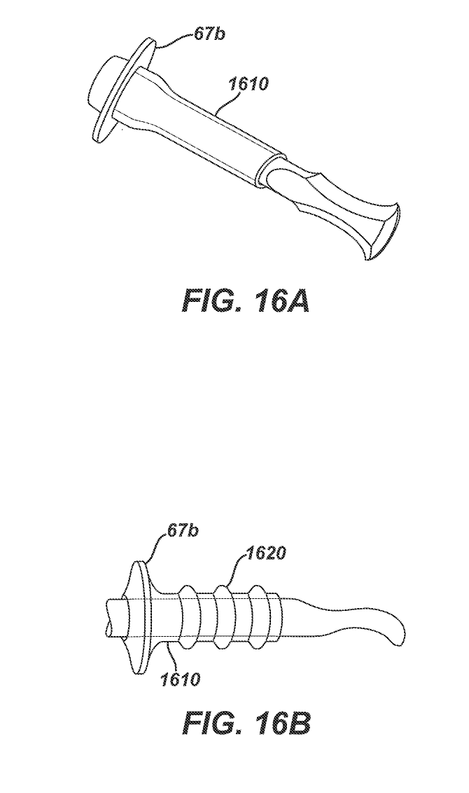

Referring now to FIG. 16A, seal 67b may be extended to cover the exposed portion of the blade 79 shaft that is not used to create the desired tissue effects in the operative field thereby creating a protective cover for the distal waveguide. The extended elastomeric material or cover 1610 may be made of varying thicknesses and in various shapes to provide the necessary bumper protection to guard against blade 79 contact with the hardware and instruments in the surgical field. The protection can take the form of a smooth surface as shown in FIG. 16A, or may be provided with ridges/bumps 1620 of varying shapes, sizes, and spacing as shown in FIG. 16B. Since the elastomeric cover 1610 is bonded directly to the blade, it does not have to have a large diameter and should not exceed the existing outer diameter of the blades protective tube, as is known in the art. This should allow the elastomeric cover 1610 to protect the blade 79 without obstructing the surgeon's view or hindering deep access of the blade 79.

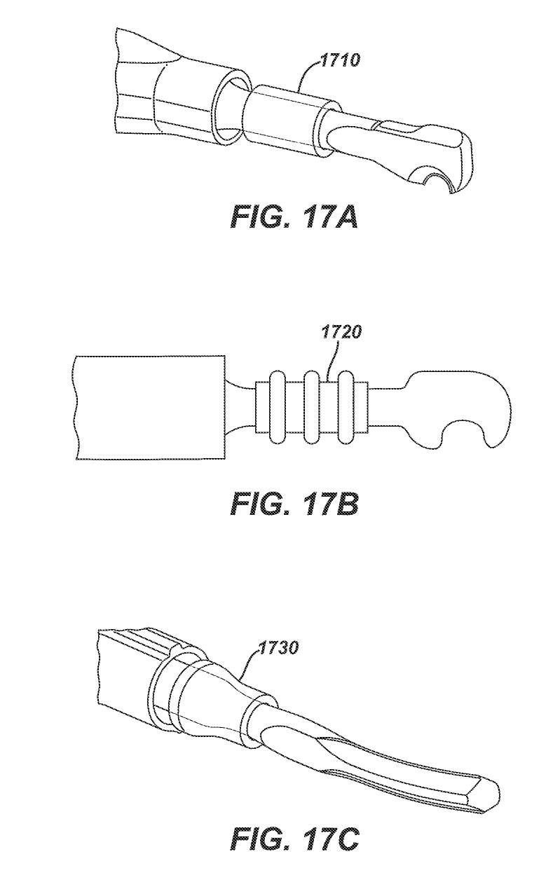

In an alternate expression of a protective elastomeric material, a single or multiple protective bumper coatings independent of any existing seals on the blade 79 may be added as shown in FIGS. 17A-17C. This coating or coatings would be placed on the exposed portion of the blade shaft that is not used to create the desired tissue effects in the operative field. The extended elastomeric material can be made of varying thicknesses and in various shapes 1710, 1720 and 1730 to provide the necessary bumper protection to guard against blade 79 contact with the hardware and instruments in the surgical field. The protection can take the form of a smooth surface 1710 or ridges/bumps 1720 of varying shapes, sizes, and spacing. The extended elastomeric material may be contiguous or may be comprised of several independent sections. Since the elastomeric overmold 1710, 1720 and 1730 is bonded directly to the blade, it does not have to have a large diameter and should not exceed the existing outer diameter of the blades protective tube thereby protecting the blade without obstructing the user's view.

Preferably, the ultrasonic apparatus 19 described above will be processed before surgery. First, a new or used ultrasonic apparatus 19 is obtained and if necessary cleaned. The ultrasonic apparatus can then be sterilized. In one sterilization technique the ultrasonic apparatus is placed in a closed and sealed container, such as a plastic or TYVEK bag. Optionally, the ultrasonic apparatus 19 can be combined in the container as a kit with other components, including a torque wrench. The container and ultrasonic device 19, as well as any other components, are then placed in a field of radiation that can penetrate the container, such as gamma radiation, x-rays, or high-energy electrons. The radiation kills bacteria on the ultrasonic apparatus and in the container. The sterilized ultrasonic apparatus can then be stored in the sterile container. The sealed container keeps the ultrasonic apparatus sterile until it is opened in the medical facility.

While the present ultrasonic device 19 has been illustrated by description of several expressions, it is not the intention of the applicants to restrict or limit the spirit and scope of the appended claims to such detail. Numerous variations, changes, and substitutions will occur to those skilled in the art without departing from the scope of the ultrasonic device. Moreover, the structure of each element associated with the present ultrasonic device can be alternatively described as a means for providing the function performed by the element. Accordingly, it is intended that the ultrasonic device be limited only by the spirit and scope of the appended claims.

We claim:

1. An ultrasonic apparatus comprising;
a waveguide having a proximal end and a distal end having a central longitudinal axis;
a blade adjacent the waveguide distal end;
the blade having a rounded distal end with a medial portion and lateral portions where the medial portion of the rounded end extends distally longer along the central axis than the lateral portions; and
the blade having concave edges adjacent to the rounded distal end lateral portions where the concave edges extend proximally along the central axis.

2. The ultrasonic apparatus of claim 1 wherein the concave edges and the rounded distal end are substantially symmetrical about the central axis.

3. The ultrasonic apparatus of claim 1 wherein a cross section of the blade has a cross section width and a central top ridge and a central bottom ridge where the blade extends laterally at obtuse angles from the central top ridge and the central bottom ridge forming the concave edges.

4. The ultrasonic apparatus of claim 2 wherein the blade rounded end curves away proximally from the central longitudinal axis.

5. The ultrasonic apparatus of claim 4 wherein the rounded end has a width greater than the cross section width of the concave edges.

6. An ultrasonic apparatus comprising:
an ultrasonic waveguide having a proximal end and a distal end defining a central axis;
an ultrasonically actuated blade attached to the distal end of the waveguide;
a housing having a proximal end and a distal end wherein the housing is adapted to be held by a user as a pencil;
a transducer disposed within the housing in mechanical communication with the waveguide;
a sheath disposed about the waveguide having a proximal end and distal end, a portion of the proximal sheath disposed within the housing distal end, the portion mechanically engaging the transducer;
a spring disposed about the sheath proximal end located substantially within the housing distal end;
a stop tooth disposed within handle; and
engagement teeth disposed about the sheath proximal end in selective mechanical communication with the stop tooth.

7. The ultrasonic apparatus of claim 6 where the blade further comprises a rounded distal end with a medial portion and lateral portions where the medial portion of the rounded end extends distally longer along the central axis than the lateral portions, the blade having concave edges proximal to the rounded distal end lateral portions where the concave edges extend proximally along the central axis.

8. The ultrasonic apparatus of claim 7 further comprising a distal waveguide cover disposed between the sheath distal end the blade.

9. The ultrasonic apparatus of claim 8 wherein the cover is comprised of elastomeric material.

10. The ultrasonic apparatus of claim 9 wherein the cover is comprised of at least two, non-contiguous sections.

11. A method of rotating an ultrasonic blade assembly, comprising:
obtaining an ultrasonic instrument, the instrument comprising
an ultrasonic waveguide having a proximal end and a distal end defining a central axis;
an ultrasonically actuated blade attached to the distal end of the waveguide;
a housing having a proximal end and a distal end wherein the housing is adapted to be held by a user as a pencil;
a transducer disposed within the housing in mechanical communication with the waveguide;
a sheath disposed about the waveguide having a proximal end and distal end, a portion of the proximal sheath disposed within the housing distal end, the sheath in rotational engagement with the transducer;
a spring disposed about the sheath proximal end located substantially within the housing distal end;
a locking tooth disposed within handle; and
engagement teeth disposed about the sheath proximal end in selective mechanical communication with the stop tooth;
applying distal longitudinal force on the sheath moving the sheath to a first position thereby disengaging the engagement teeth from the stop tooth;
applying a rotational force to the sheath while the sheath is in the first position thereby rotating the sheath and the blade; and
releasing the sheath, the spring biasing the sheath proximally to engage engagement teeth with the stop tooth, thereby preventing further rotation.

12.
